(19) United States
(12) Patent Application Publication
(10) Pub. No.: US 2005/0129637 A1
(43) Pub. Date: Jun. 16, 2005

(54) Pigment dispersion for cosmetics, cosmetic compositions containing the same, and process for making such cosmetic compositions

(76) Inventor: Gakushi Aota, Hyogo (JP)

Correspondence Address: YOUNG & THOMPSON, 745 SOUTH 23RD STREET, 2ND FLOOR, ARLINGTON, VA, 22202, US

(21) Appl. No.: 10/735,670

(22) Filed: Dec. 16, 2003

(51) Int. Cl. A61K 7/021

(52) U.S. Cl. 424/63

(57) ABSTRACT

In pigment dispersions for cosmetic products, at least 90 vol % of pigment particles have a dispersed diameter of at most 0.7 μm. The pigment dispersions preferably also contain a dispersing medium and a dispersant. The dispersing medium may be an aqueous medium. A process for producing cosmetic compositions includes adding a pigment dispersion as described above to a cosmetic material as coloring agent. The pigment dispersion may be added to a polymer emulsion and/or a thickener.

### BACKGROUND OF THE INVENTION

The entire contents of Japanese Application No. 2001-208557, filed on Jun. 6, 2001, are hereby incorporated by reference.

1. Field of the Invention

This invention relates to pigment dispersions for cosmetic products, cosmetic compositions containing the same, and a process for preparing such cosmetic compositions.

2. Description of Related Art

In general, pigments to be used in makeup cosmetics such as eyeliner, eyebrow pencil, mascara, lipstick, nail polish, and the like, are mixed with the other ingredients in the form of a powder or melted particles, then dispersed by known dispersing machines such as a ball mill, sand mill, bead mill, roll mill, homomixer, attrition mill, etc.

For instance, in Japanese Patent No. 2604190, it is disclosed that a dispersion obtained by dispersing iron oxide particles having a certain size with a polymer dispersant and water in a ball mill or a dino-mill, has good permeability to the applied surface when used in an applicator type container.

Also, in Japanese Kokai No. 11-189,513, inorganic coloring particles surface-treated with alumina are added to water with surfactant and antifoaming agent, and the obtained aqueous dispersion is mixed with polymer emulsion, to produce an aqueous makeup cosmetic with improved prevention of color separation, a smooth feeling to the touch, and that continues to look good even after being worn for a prolonged time. It is also known that a pigment for cosmetic products can be wet-milled, and in Japanese Kokai No.4-103,516, it is disclosed that an aqueous nail polish can be obtained by wet-milling the pigment with an aqueous polymer emulsion as dispersant.

However, by using the known methods as described above, the pigments were not sufficiently dispersed, or were overly dispersed, and therefore the obtained particle size distribution was broad, and a lot of coarse particles remained. Also, the coloration or the luster of the obtained cosmetic product, and the stability overtime was not sufficient. By simply dispersing pigment in advance in various kinds of dispersing media, and then combining the previously-prepared dispersion with the other ingredients to obtain a cosmetic product, pigment particles agglomerate, and the dispersed state is not satisfactory. Especially in an aqueous medium, it was very difficult to obtain a stable aqueous dispersion in which pigment particles are dispersed in a very fine state.

It is also known to wet-mill the pigment in an aqueous medium, but by simply wet-milling, the obtained dispersed state was not satisfactory, that it, the pigment was not thoroughly dispersed or was overly dispersed, and the particle size distribution was broad, and a lot of coarse particles remained, and the coloration or luster, and the stability during the passage of time was not satisfactory.

### SUMMARY OF THE INVENTION

The present inventor has conducted an extensive study in view of the above problems. As a result, he has discovered that a pigment dispersion having a certain particle size distribution has excellent coloration and luster, and good stability over time. The present invention has been attained based on this finding.

Thus, the present invention provides pigment dispersions for cosmetic products, in which at least 90 vol % of pigment particles have a dispersed diameter of at most 0.7 μm. The pigment dispersions preferably also contain a dispersing medium and a dispersant. The dispersing medium may be an aqueous medium.

A process for producing cosmetic compositions according to the invention includes adding a pigment dispersion as described above to a cosmetic material as coloring agent. The pigment dispersion may be added to a polymer emulsion and/or a thickener.

Cosmetic compositions according to the invention contain at least one pigment, in which 90 vol % of pigment particles have a dispersed diameter of at most 0.7 μm. Such cosmetic compositions may also include a dispersing medium and a dispersant. The dispersing medium may be an aqueous medium.

### DETAILED DESCRIPTION OF PREFERRED EMBODIMENTS

Preferred embodiments of the invention will now be described in greater detail, to aid in understanding the invention.

The pigment that can be used in the present invention is not particularly limited. Various kinds of inorganic pigments and organic pigments may both be used, singly or in combination. Typical examples of the inorganic pigment are, for instance: oxides such as titanium dioxide, zinc oxide, chromium oxide, black oxide of iron, red oxide of iron, yellow oxide of iron, and iron oxide titanium oxide sintered products.

Typical examples of the inorganic pigment are, for instance: coloring pigments such as prussian blue, ultramarine, and carbon black.

More specific examples of the organic pigment are, for instance: aluminum, barium or zirconium lake of pigment such as amaranth (C.I. 16185), erythrosine (C.I. 45430), newcoccin (C.I. 16255), phloxine B (C.I. 45410), rose Bengal (C.I. 45440), acid red (C.I. 45100), tartrazine (C.I. 19140), sunset yellow FCF (C.I. 15985), fastgreen FCF (C.I. 42053), brilliant blue FCF (C.I. 42090), indigo carmine (C.I. 73015); calucium, barium, strontium lake of lithol rubine B (C.I. 15850); lithol rubine BCA (C.I. 15850), lake red C (C.I. 15585), lake red BCA (C.I. 15585), deep maroon (C.I. 15880), tetrabromofluorescein (C.I. 45380), sudan III (C.I. 26100), helindone pink CN (C.I. 73360), permaton red (C.I. 12085), benzidine orange G (C.I. 21110), benzidine yellow G (C.I. 21090), carbanthrene blue (C.I. 69825), alizurine purple SS (C.I. 60725), brilliant fast scarlet (C.I. 12315), hanza orange (C.I. 11725), hanza yellow (C.I. 11680), phtalocyanine blue (C.I. 74160).

Especially recommended inorganic pigments are, for instance: titanium dioxide, zinc oxide, black oxide of iron, red oxide of iron, yellow oxide of iron, titanium black, prussian blue, and carbon black. Especially recommended organic pigments are, for instance: calcium, barium or strontium lake of lithol rubine B (C.I. 15850); lithol rubine BCA (C.I. 15850), deep maroon (C.I. 15880), helindone pink CN (C.I. 73360), benzidine orange (C.I. 21110), benzidine yellow G (C.I. 21090), carbanthrene blue (C.I. 69825), hanza yellow (C.I. 11680), and phtalocyanine blue (C.I. 74160).

Especially by using one or more among these pigments, by using them as in the particle range described in the present invention, the improvement of properties such as coloration are conspicuous.

The amount of the pigment in the dispersion is preferably 1 to 40 wt % as solid content, more preferably 10 to 30 wt %. In this range the dispersion has an especially good handling property and is suitable for the production of various cosmetic products.

The expression of physical parameters as numerical values in this specification and in the appended claims is understood not to be limited to the exact numerical values expressed, but rather encompasses such variations above and below the stated values as occur in practice due to insubstantial manufacturing variations and measuring and/or rounding effects and imprecision.

The dispersion medium that can be used in the present invention is not particularly limited. Various kinds of media may be used, but use of a dispersion medium that contains water is recommended, to obtain an aqueous dispersion that is suitable as coloring material for various aqueous cosmetic products. Here, an aqueous dispersion connotes a dispersion containing at least 50 wt % of water. A dispersion of the present invention has a very high stability of the dispersed state, therefore, it can contain even more than 60 wt % or more than 70 wt % of water. It may be a dispersion that contains no organic solvent.

The pigment dispersion of the present invention may have an improved stability of the dispersed state, by using a dispersant. Here, a dispersant that can be used is not limited, and for example, water-soluble polymers, or various kinds of surfactants can be used.

Specific examples of the water-soluble polymer are: styrene/acrylic acid copolymer, styrene/methacrylic acid copolymer, styrene/-methyl styrene/acrylic acid copolymer, acrylic acid/alkyl copolymer, acrylic acid ester/methacrylic acid copolymer, styrene/maleic acid copolymer, methoxyethylene/anhydric maleic acid copolymer and its half ester, polyacrylic acid, polyaspara-acid, polyglutamic acid, etc., may be used. Also, their salts such as alkyl salts, sodium salts, potassium salts, lithium salts; ammonium salts; and alkanol salts such as mono-, di-, tri-ethanol amine, triisopropanol amine, may be used.

Other concrete examples of the water-soluble polymer are: polyvinyl pyrrolidone/vinyl acetate copolymer, polyvinyl pirrolydone, may be used.

Examples of the anionic surfactant are: soap base, zinc laurate, zinc myristate, magnesium myristate, zinc palmitate, magnesium stearate, zinc stearate, calcium stearate, sodium lauric sulfuric acid, lauric sulfuric acid triethanol amine, sodium cetyl sulfuric acid, polyoxyethylene lauric ether sulfuric acid triethanol amine, sodium polyoxyethylene lauric ether surfuric acid, polyoxyethylene lauric ether phosphoric acid, sodium polyoxyethylene lauric ether phosphoric acid, polyoxyethylene cetyl ether phosphoric acid, sodium polyoxyethylene cetyl ether phosphoric acid, polyoxyethylene stearic ether phosphoric acid, polyoxyethylene oleic ether phosphoric acid, sodium polyoxyethylene oleic ether phosphoric acid, polyoxyethylene alkyl (12-16)ether phosphoric acid, polyoxyethylene alkyl phenyl ether phosphoric acid, polyoxyethylene alkyl phenyl ether phosphoric acid triethanol amine, sodium polyoxyethylene alkyl phenyl ether phosphoric acid, sodium sulfo succinate dioctyl, etc.

Examples of the nonionic surfactant are: monostearic acid glyceride, monooleic acid glyceride, monostearic acid ethylene glycol, monostearic acid propylene glycol, dioleic acid propylene glycol, monolauric acid sorbitane, monopalmitic acid solbitane, monostearic acid solbitane, monooleic acid solbitane, sesquioleic acid solbitane, trioleic acid solbitane, sucrose fatty acid ester, polyoxyethylene lauric ether, polyoxyethylene stearyl ether, polyoxyethylene octyl phenyl ether, polyoxyethylene nonyl phenyl ether, polyoxyethylene octyl dodecyl ether, polyoxyethylene isocetyl ether, polyoxyethylene isostearyl ether, polyoxyethylene oleic cetyl ether, polyoxyethylene stearic acid amid, monostearic acid polyoxyethyleneglycerin, monolauric acid polyoxyethylene sorbit, monooleic acid polyoxyethylene sorbit, trioleic acid polyoxyethylene sorbitan, tetraoleic acid polyoxyethylene sorbit, polyoxyethylene castor oil, polyoxyethylene hardened castor oil, polyoxyethylene lanorin.

Among these dispersants, especially styrene/acrylic acid copolymer, styrene/α-methylstyrene/acrylic acid copolymer, polyacrylic acid, polyglutamic acid, polyaspartic acid, and more preferably their sodium salts and their ammonium salts, and polyoxyethylene lauric ether phosphoric acid, sodium polyoxyethylene lauryl ether phosphoric acid, polyoxyethylene cetyl ether phosphoric acid, sodium polyoxyethylene cetyl ether phosphoric acid, polyoxyethylene stearyl ether phosphoric acid, polyoxyethylene oleil ether phosphoric acid, sodium polyoxyethylene oleil ether phosphoric acid, polyoxyethylene alkyl (12-16) ether phosphoric acid, polyoxyethylene alkyl phenyl ether phosphoric acid, etc. anionic surfactants, polyoxyethylene lauryl ether, polyoxyethylene oleyl ether, polyoxyethylene cetyl ether, polyoxyethylene stearyl ether, polyoxyethylene octyl pheny ether, polyoxyethylene nonyl phenyl ether, polyoxyethylene octyl dodecyl ether, polyoxyethylene isocetyl ether, polyoxyethylene iso stearyl ether, polyoxyethylene oleyl cetyl ether, etc. polyoxyethyelen type nonionic surfactants are prefered.

One or more of these dispersants may be used in combination according to the intended use of the products.

The ratio of the dispersants are preferably from 0.1 to 40 parts by weight, more preferably from 10 to 35 parts by weight, based on 100 parts by weight of pigment.

Various kinds of water-soluble solvent may be included, such as polyethylene glycol, glycerin, propylene glycol, ethylene glycol, isoprene glycol, 1,3-butylene glycol.

Further additives such as humectants, preservatives, pH-adjusters may be included. As humectants, there are for example, ethylene glycol, diethylene glycol, triethylene glycol, propylene glycol, dipropylene glycol, polyprolylene glycol, 1,3-prolylene glycol, isoprene glycol, polyethylene glycol, hexylene glycol, glycerin, concentrated glycerin, diglycerin, polyglycerin, sorbitol, sorbitol syrup, maltitol.

As preservatives, there are for example, chlorobuthanol, chlorcresol, parachlormethaxylenol, cresol, trichlosan, trichlorocarbanide, isopropylmethylphenol, benzethonium chloride, benzethonium chloride solution, cetylpyridinium chloride, thianthol, phenol, paraphenol sodium sulfuric acid (dihydrate), paraphenol zinc sulfuric acid, resorcin, Photosensitizing Dye No. 101, 201, 301, 401, Hinokitiol, 1-menthol, dl-menthol, d-camphor, dl-camphor, benzoic acid, sodium benzoate, paraoxy zenzoate ester, sorbic acid, potassium sorbate, dehydroacetic acid, sodium dehydroacetate, salicylic acid, sodium salicylate, methyl salicylate, phenyl salicylate, etc.

As pH-adjusters, there are for example, monoethanolamine, diethanolamine, triethanolamine, isopropanolamine, diisopropanolamine, triisopropanolamine, 2-amino-2-methyl-1-propanol, 2-amino-2-methyl-1,3-propanediol, morpholine, sodium hydroxide, magnesium hydroxide, potassium hydroxide, Strong Ammonia Solution.

In a pigment dispersion of the present invention for cosmetic product, besides the above-mentioned ingredients, the ingredients that may be included in the cosmetic products, for example, thickeners, chelating agents, antifoaming agents, may be added as desired. However, as a pigment dispersion of the present invention has a very stable dispersed state, it can be a dispersion with very low viscosity, without adding any thickener.

A process for producing a pigment dispersion of the present invention is not particularly limited. A typical method for producing a pigment dispersion of the present invention is conducted, for instance, by blending the ingredients above mentioned, and then wet-milling using any of the known dispersing devices such as a ball mill, sand mill, roll mill, or homomixer. During this process, the particle size distribution is checked at adequate time so that at least 90 vol % of the pigment particles have diameter of not more than 0.7 μm, and to conduct the milling process until at least 90 vol % of the pigment particle are not more than 0.7 μm. To conduct this process, the adequate amount of dispersant is defined for each of pigment species, and the speed of rotation and shear force is adequately adjusted, so that the required particle distribution is obtained.

To check the particle size distribution, “Microtrack UPA MODEL:9340-UPA”(Nikkiso Corp.) may be suitably used. That at least 90 vol % of the pigment particles have diameter of not more than 0.7 μm can also be described in other words that the D₉₀ of the dispersion is 0.7 μm.

A pigment dispersion of the present invention, which may be obtained by the method described above, exhibits satisfying quality in the properties such as the average particle size at room temperature and at 50° C., pH, viscosity, particle distribution, hue, and precipitation.

As above mentioned, a pigment dispersion of the present invention is characterized in that at least 90 vol % of the particles have a dispersed size not more than 0.7 μm. Here, the dispersed size of the pigment particle is the size of the pigment particle in the liquid medium of the dispersion, that can be measured by the method described above. Namely, by using means such as “Microtrack UPA MODEL:9340-UPA” (Nikkiso Corp.) as apparatus, analyzing the diluted sample obtained by diluting the sample with the ion exchanged water so that the signal level is from 0.1 to 100, preferably from 0.6 to 80.

That at least 90 vol % of the particles have the dispersed size not more than 0.7 μm means that by measurement using the above mentioned apparatus and the method, the particle size distribution of the pigment in the dispersion is obtained, and the particle size on the cumulative curve of the distribution of particle diameter in the dispersion at the point corresponding to 90 vol % of total particles of the obtained particle distribution, which is usually indicated as D₉₀ (90 volumetric percent diameter), is not more than 0.7 μm.

If the particle with diameter not more than 0.7 μm is less than 90 vol %, because of the coarse particles, coloration or luster maybe deteriorated, pigment particles tend to agglomerate, and precipitation or sedimentation of the pigment particles occurs during the passage of time, thus a dispersion with good stability during the passage of time is difficult to obtain.

Furthermore, the pigment dispersion of the present invention may have the 90 vol % diameter (D₉₀) of not more than 0.5 μm, or even not more than 0.4 μm.

The mean particle size of the pigment of a dispersion of the present invention is not limited, but it is preferably not more than 0.5 μm, more preferably not more than 0.3 μm. By having the mean particle size in this range, a pigment dispersion able to present cosmetic products having an especially good dispersing stability and coloration and luster can be obtained.

The viscosity of the pigment dispersion of the present invention is not limited, but the pigment dispersion of the present invention may have a very low viscosity, for instance, not more than 5 cps, or even not more than 2 cps, or even not more than 1 cps. Therefore, its handling, and blending with other ingredients of the cosmetic products are easy. In a case which a higher viscosity is preferred, it can be adjusted by adding thickeners.

By using the pigment dispersion above described as colorant, a cosmetic product may be obtained. For instance, a pigment dispersion of the present invention is blended with polymer emulsion, thickener, etc., an aqueous cosmetic product such as eyeliner, eyebrow, mascara, lipstick, nail polish, can be obtained.

As polymer emulsion, typically, there are such water-soluble polymer emulsion as: acrylic acid resin type, styrene/acrylic acid resin type, vinyl acetate type, styrene/butadiene resin type, polyurethane type, olefin resin type, alkyd resin type, etc. As thickener, there are: methyl cellulose, carboxymethyl cellulose, hydroxyethyl cellulose, hydroxypropyl cellulose, polyvinyl alcohol, polyvinyl pyrrolidone, carboxyvinyl polymer, sodium polyacrylic acid, polyethylene glycol, oxyethylene/oxypropylene block copolymer, gum arabic, alginic acid sodium salt, xanthan gum, casein, cyamoposis gum, locust bean gum, bentonite type thickener.

In an aqueous cosmetic product thus obtained, the pigment particles may be dispersed in a very fine and homogeneous state. Therefore, properties such as coloration and luster may be improved. Furthermore, a dispersion of the present invention may be obtained as a pigment dispersion containing no organic solvent, which leads to the advantages such that no harm to the living bodies, inflammability, solvent smell, are to be concerned.

Furthermore, in a cosmetic product obtained by using a pigment dispersion of the present invention, at least 90 vol % of the pigment particles may have a dispersed diameter size of not more than 0.7 μm.

### EXAMPLES

The present invention is further illustrated by the following examples of the invention.

### Example 1

| component | amount (parts by weight) |
|---|---|
| purified water | 68.4 |
| acrylic acid/alkyl styrene copolymer | 5.0 |
| strong ammonia solution | 0.2 |
| 1.3-butylene glycol | 5.0 |
| phenoxyethanol | 0.7 |
| methyl p-hydroxybenzoate | 0.3 |
| isopropyl p-hydroxybenzoate | 0.1 |
| disodium edetate | 0.1 |
| 2-amino-methyl-1-propanol | 0.2 |
| phthalocyanine blue (C.I. 74160) | 20.0 |

The above components were mixed by a bead-mill, by verifying the particle distribution with “Microtrack UPA MODEL:9340-UPA” (Nokkiso Corp.), and a pigment dispersion with more than 90 vol % of the particles having dispersed diameter of not more than 0.37 μm was obtained. This pigment dispersion had pigment average dispersed diameter of 0.12 μm, and its viscosity was 6 cps. After 3 months of standing still, this pigment showed no precipitate. Also, both at room temperature and at 50° C., average dispersed diameter, pH, particle distribution of pigment particle, and hue, were stable after 3 months.

### Example 2

| component | amount (parts by weight) |
|---|---|
| purified water | 75.5 |
| polyoxyethylene alkyl (12-16) ether | 3.0 |
| phosphoric acid | |
| polyoxyethylene oleyl ether | 0.4 |
| sodium hydroxide | 0.15 |
| 1,3-butylene glycol | 5.0 |
| phenoxy ethanol | 0.7 |
| methyl p-hydroxybenzoate | 0.3 |
| lithol rubine BCA (C.I. 15850) | 20.0 |

The above ingredients were mixed, and by verifying the particle distribution with “Microtrack UPA MODEL:9340-UPA” (Nokkiso Corp.), it was milled in a bead mill, and a pigment dispersion with more than 90 vol % of the particles having dispersed diameter of not more than 0.36 μm was obtained. This pigment dispersion had pigment average dispersed diameter of 0.15 μm, and its viscosity was 4 cps. After 3 months of standing still, this pigment dispersion showed no precipitation. Also, both at room temperature and at 50° C., average dispersed diameter, pH, particle distribution of pigment particle, and hue were stable after 3 months.

### Example 3

| component | amount (parts by weight) |
|---|---|
| purified water | 80.6 |
| polyvinyl pyrrolidone | 3.0 |
| 1,3-butylene glycol | 5.0 |
| phenoxyethanol | 0.7 |
| methyl p-hydroxybenzoate | 0.3 |
| isopropyl p-hydroxybenzoate | 0.1 |
| disodium edetate | 0.1 |
| 2-amino-methyl 1-propanol | 0.2 |
| carbon black | 10.0 |

The above ingredients were mixed, and by verifying the particle distribution with “Microtrack UPA MODEL:9340-UPA” (Nokkiso Corp.), it was milled in a bead mill, and a pigment dispersion with more than 90 vol % of the particles having dispersed diameter of not more than 0.28 μm was obtained. This pigment dispersion had pigment average dispersed diameter of 0.16 μm, and its viscosity was 4 cps. After standing still for 3 months, this pigment dispersion showed no precipitation. Also, both at room temperature and at 50° C., average dispersed diameter, pH, particle distribution of pigment particle, and hue were stable after 3 months.

### Example 4

| component | amount (parts by weight) |
|---|---|
| purified water | 66.0 |
| acrylic acid/alkyl styrene copolymer | 2.0 |
| strong ammonia solution | 0.6 |
| 1,3-butanediol | 5.0 |
| phenoxyethanol | 0.7 |
| methyl p-hydroxybenzoate | 0.3 |
| isopropyl p-hydroxybenzoate | 0.1 |
| disodium edetate | 0.1 |
| 2-amino-methyl 1-propanol | 0.2 |
| titanium black | 25.0 |

The above ingredients were mixed, and by verifying the particle distribution with “Microtrack UPA MODEL:9340-UPA” (Nokkiso Corp.), it was milled in a bead mill, and a pigment dispersion with more than 90 vol % of the particles having dispersed diameter of not more than 0.65 μm was obtained. This pigment dispersion had pigment average dispersed diameter of 0.26 μm, and its viscosity was 3.8 cps. After standing still for 3 months, this pigment dispersion showed no precipitation. Also, both at room temperature and at 50° C., average dispersed diameter, pH, particle distribution of pigment particle, and hue were stable after 3 months.

### Comparative Example 1

| component | amount (parts by weight) |
|---|---|
| purified water | 72.0 |
| polyvinyl pyrrolidone | 3.0 |
| 1,3-butanediol | 5.0 |
| phenoxyethanol | 0.7 |
| methyl p-hydroxybenzoate | 0.3 |
| helindone pink CN (C.I. 73360) | 20.0 |

The above ingredients were mixed, and by verifying the particle distribution with “Microtrack UPA MODEL:9340-UPA” (Nokkiso Corp.), it was milled in a bead mill, and a pigment dispersion with more than 40 vol % of the particles having dispersed diameter of more than 6 μm was obtained. This pigment dispersion separated into two layers after 1 hour of standing still.

### Comparative Example 2

| component | amount (parts by weight) |
|---|---|
| purified water | 64.0 |
| 1,3-butanediol | 5.0 |
| phenoxyethanol | 0.7 |
| methyl p-hydroxybenzoate | 0.3 |
| acrylic acid alkanol amine liquid | 10.0 |
| benzidine yellow (C.I. 21090) | 20.0 |

The above ingredients were mixed, and by verifying the particle distribution with “Microtrack UPA MODEL:9340-UPA” (Nokkiso Corp.), it was milled in a bead mill, and a pigment dispersion with more than 40 vol % of the particles having dispersed diameter of more than 6 μm was obtained. This pigment dispersion separated into two layers after 1 hour of standing still.

As demonstrated above, the pigment dispersions of the present invention have a very finely dispersed and stable dispersed state, and excellent coloration and luster, suitable for use as colorant for various cosmetic products. The cosmetic compositions of the present invention using such pigment dispersions likewise have very finely dispersed and stable dispersed state, and excellent coloration and luster, suitable for various cosmetic products.

While the present invention has been described above in connection with various preferred embodiments, it is to be understood that these embodiments are provided for the purpose of illustrating the invention, and should be viewed in a limiting sense. Persons skilled in this art upon reading this specification will envision a variety of additional embodiments within the scope off the invention set forth herein, and such additional embodiments as well as all insubstantial variations on the invention as set forth herein should be regarded as falling within the true scope and spirit of the appended claims.

1. A pigment dispersion for cosmetic products, wherein at least 90 vol % of pigment particles have a dispersed diameter of at most 0.7 μm.

2. The pigment dispersion according to claim 1, comprising at least one pigment, a dispersing medium and a dispersant.

3. The pigment dispersion according to claim 1, wherein the dispersing medium is an aqueous medium.

4. The pigment dispersion according to claim 2, wherein the dispersing medium is an aqueous medium.

5. A process for producing a cosmetic composition, comprising combining with at least one cosmetic ingredient a pigment dispersion according to claim 1 as coloring agent.

6. The process according to claim 5, wherein the pigment dispersion is a pigment dispersion according to claim 2.

7. The process according to claim 5, wherein the pigment dispersion is a pigment dispersion according to claim 3.

8. The process according to claim 5, wherein the pigment dispersion is a pigment dispersion according to claim 4.

9. A process for producing a cosmetic composition, comprising combining the pigment dispersion according to claim 1 with a polymer emulsion and a thickener.

10. The process according to claim 9, wherein the pigment dispersion is a pigment dispersion according to claim 2.

11. The process according to claim 9, wherein the pigment dispersion is a pigment dispersion according to claim 3.

12. The process according to claim 9, wherein the pigment dispersion is a pigment dispersion according to claim 4.

13. A cosmetic composition comprising at least one pigment and at least one cosmetic ingredient, wherein at least 90 vol % of pigment particles have a dispersed diameter of at most 0.7 μm.

14. The cosmetic composition according to claim 13, further comprising a dispersing medium and a dispersant.

15. The cosmetic composition according to claim 14, wherein the dispersing medium is an aqueous medium.
